(19) United States
(12) Patent Application Publication
(10) Pub. No.: US 2026/0198962 A1
(43) Pub. Date: Jul. 16, 2026

(54) PUNCTURE NEEDLE, PUNCTURE CATHETER PROVIDED WITH PUNCTURE NEEDLE, AND CATHETER SYSTEM PROVIDED WITH PUNCTURE CATHETER

(71) Applicants: KANEKA CORPORATION, Osaka (JP); Kyoto University, Kyoto (JP)

(72) Inventors: Hirokazu Kamakura, Nagano (JP); Yuki Mukai, Nagano (JP); Kohei Fukaya, Nagano (JP); Shin Watanabe, Kyoto (JP); Ryusuke Nishikawa, Kyoto (JP); Munekazu Tanaka, Kyoto (JP)

(73) Assignees: KANEKA CORPORATION, Osaka (JP); Kyoto University, Kyoto (JP)

(21) Appl. No.: 19/558,861

(22) Filed: Mar. 6, 2026

Related U.S. Application Data
(63) Continuation of application No. PCT/JP2024/028421, filed on Aug. 8, 2024.

(30) Foreign Application Priority Data
Sep. 7, 2023 (JP) 2023-145174
Sep. 7, 2023 (JP) 2023-145176
Sep. 7, 2023 (JP) 2023-145177
Sep. 7, 2023 (JP) 2023-145178
Sep. 7, 2023 (JP) 2023-145179

(51) Int. Cl. A61B 17/34; A61M 25/06

(52) U.S. Cl. CPC A61B 17/3415; A61M 25/06

(57) ABSTRACT

A puncture needle is a medical puncture needle having a longitudinal direction and including a tubular member having an inner cavity extending in the longitudinal direction of the puncture needle and a helical member in which a wire is wound helically around the tubular member. A distal end portion of the wire and the tubular member are not fixed to each other, or the distal end portion of the wire and the tubular member are not in contact with each other. A puncture catheter includes the puncture needle, and a catheter system includes the puncture catheter.

### CROSS-REFERENCE TO RELATED APPLICATIONS

This application claims priority to Japanese Patent Application Nos. 2023-145174, 2023-145176, 2023-145177, 2023-145178, and 2023-145179 filed on Sep. 7, 2023, the entire contents of which are incorporated herein by reference.

### TECHNICAL FIELD

One or more embodiments of the present invention relate to a medical puncture needle, a puncture catheter including the puncture needle, and a catheter system including the puncture catheter.

### BACKGROUND

Therapies and the like that involve directly administering a drug solution such as a myocardial regenerative cell preparation to cardiac muscle cells that are losing the functions thereof owing to a myocardial infarction or the like so as to regenerate the cardiac muscle cells are performed. In a case where such a drug solution is directly administered to an organ inside a body in this manner, a catheter having a needle needs to be inserted into a body cavity, and the needle needs to be caused to puncture the organ. As needles for use in such treatments, for example, the following needles have been developed.

Patent Literature 1 describes a drug solution injection needle that is a hollow needle for puncturing the myocardium of a patient and injecting a drug solution thereinto, the drug solution injection needle including: a sharp tip member formed from a metal; an electrically insulative connection tube connected to a proximal end side of the tip member; a metal tube connected to a proximal end side of the connection tube; and an insulating layer coating an outer circumferential surface of a proximal end portion of the metal tube. The connection tube and/or the tip member has an outer surface in which at least one hole (an outflow path for the drug solution) is opened, the at least one hole being formed in communication with an inner cavity of the needle. The metal tube has a distal end portion that is not coated with the insulating layer and that serves as an electrode for measuring a potential.

Patent Literature 2 describes a catheter including a helical needle for puncturing the myocardium of a patient and injecting a drug solution thereinto, the catheter including: a catheter body having a distal end and a proximal end and having a delivery lumen extending through these ends; and a helical needle having a helical delivery lumen connected so as to receive an injectable substance from the delivery lumen of the catheter body. It is also disclosed that the helical needle punctures the myocardium by being rotated.

### PATENT LITERATURE

PTL 1: International Publication No. WO2021/192283
PTL 2: Japanese Laid-Open Patent Publication (Translation of PCT Application) No. 2016-537040

However, the needle described in Patent Literature 1 easily comes out of tissue after puncture and has difficulty in finely adjusting a puncture depth thereof.

The needle described in Patent Literature 2 has difficulty in puncturing the target tissue since a central axis thereof becomes unstable during rotation.

### SUMMARY

One or more embodiments of the present invention have been made in view of the above, and a puncture needle that easily punctures a target tissue, is difficult to come out of the target tissue, and allows easy adjustment of a puncture depth thereof, a puncture catheter including the puncture needle, and a catheter system including the puncture catheter are provided.

A puncture needle according to one or more embodiments of the present invention is as follows.
[1] A medical puncture needle having a longitudinal direction, the puncture needle comprising:
a tubular member having an inner cavity extending in the longitudinal direction; and
a helical member in which a wire is wound helically around the tubular member, wherein
a distal end portion of the wire and the tubular member are not fixed to each other, or the distal end portion of the wire and the tubular member are not in contact with each other.

Since the helical member is disposed around the tubular member in the puncture needle, the puncture needle can be easily screwed into a target tissue by rotating the puncture needle about the tubular member as an axis. Accordingly, it becomes easier for the puncture needle to puncture the target tissue. In addition, the wire constituting the helical member bites into the tissue, so that it becomes difficult for the puncture needle to come out of the tissue. Furthermore, the puncture depth of the puncture needle can be easily adjusted by adjusting the rotation of the puncture needle.

The puncture needle according to one or more embodiments of the present invention may be any of [2] to [18] below.
[2] The puncture needle according to above [1], wherein an outer surface of the tubular member and an inner surface of the helical member are separated from each other in a section in which a distal portion of the helical member is located in the longitudinal direction.
[3] The puncture needle according to above [2], wherein the outer surface of the tubular member and the inner surface of the helical member are separated from each other due to presence of a gap between the outer surface of the tubular member and the inner surface of the helical member in the section in which the distal portion of the helical member is located in the longitudinal direction.
[4] The puncture needle according to any one of above [1] to [3], wherein a cross-sectional shape of the wire is polygonal in a cross-section perpendicular to the longitudinal direction.
[5] The puncture needle according to any one of above [1] to [4], wherein, in a cross-section perpendicular to the longitudinal direction and passing through a midpoint of the puncture needle in the longitudinal direction,
when a length in a radial direction of the tubular member between the outer surface of the tubular member and a portion of the wire located outermost in the radial direction of the tubular member is denoted by H,
a separation distance between the outer surface of the tubular member and the inner surface of the helical member is denoted by h, and
a width of the wire in a circumferential direction of the puncture needle is denoted by W,
relationships of 2h<H and H−h<W are satisfied.
[6] The puncture needle according to any one of above [1] to [5], wherein a distal end of the helical member is located on a proximal side with respect to a distal end of the tubular member.
[7] The puncture needle according to any one of above [1] to [6], wherein a hole providing communication between the inner cavity of the tubular member and an exterior of the puncture needle is formed in the tubular member.
[8] The puncture needle according to any one of above [1] to [7], wherein the tubular member includes: a tapered portion having an outer diameter decreasing toward a distal side; and a straight tube portion located on a proximal side with respect to the tapered portion.
[9] The puncture needle according to any one of above [1] to [8], wherein the wire has a solid structure.
[10] The puncture needle according to any one of above [1] to [9], configured to puncture an organ inside a body.
[11] The puncture needle according to any one of above [1] to [10], wherein a part of the tubular member and a part of the helical member are directly fixed to each other.
[12] The puncture needle according to above [7], wherein
the tubular member has a wire-arrangement region in which the wire is disposed radially outward of the tubular member and a wire-non-arrangement region in which the wire is not disposed radially outward of the tubular member, and
the hole is formed in the wire-non-arrangement region of the tubular member.
[13] The puncture needle according to above [12], wherein the hole is not formed in the wire-arrangement region of the tubular member.
[14] The puncture needle according to any one of above [1] to [13], wherein an inner diameter of the helical member is equal to or less than twice an outer diameter of the tubular member.
[15] The puncture needle according to any one of above [1] to [14], wherein the distal end portion of the wire has a tapered shape.
[16] The puncture needle according to any one of above [1] to [15], wherein, in a cross-section perpendicular to the longitudinal direction and passing through a midpoint of the puncture needle in the longitudinal direction, a length of the wire in a radial direction of the tubular member is shorter than a length of the wire in a circumferential direction of the tubular member.
[17] The puncture needle according to any one of above [1] to [16], wherein the wire has a section in which a value obtained by dividing a length of the wire in a circumferential direction of the tubular member, measured in a cross-section perpendicular to the longitudinal direction, by a length of the wire in a radial direction of the tubular member, measured in the cross-section, decreases toward a distal side.
[18] The puncture needle according to above [15], wherein the tapered shape extends from a distal end of the wire to a position away from the distal end of the wire on a proximal side by a length equal to ½ of a pitch of the helical member.

One or more embodiments of the present invention also include the following puncture catheter.
[19] A puncture catheter comprising the puncture needle according to any one of above [1] to [18].
[20] The puncture catheter according to above [19], further comprising a first tube having an inner cavity extending in the longitudinal direction and connected to a proximal end portion of the puncture needle, wherein
the first tube has a plurality of grooves formed therein.
[21] The puncture catheter according to above [20], wherein the first tube is made of a metal.
[22] The puncture catheter according to above [20] or [21], wherein a first resin layer made of a resin is provided on an outer surface of the first tube.
[23] The puncture catheter according to any one of above [20] to [22], wherein an extending direction of the grooves and the longitudinal direction intersect each other at an angle α when observed in a direction perpendicular to the longitudinal direction.
[24] The puncture catheter according to above [23], wherein the angle α is 70° or more and 85° or less.
[25] The puncture catheter according to any one of above [20] to [24], wherein
the plurality of grooves include a first groove and a second groove located at a position different from that of the first groove, and
the second groove is located on an extending direction of the first groove when observed in a direction perpendicular to the longitudinal direction.
[26] The puncture catheter according to above [25], wherein a maximum length between the first groove and the second groove in the extending direction of the first groove is shorter than a maximum length of the first groove in the extending direction of the first groove.
[27] The puncture catheter according to above [25] or [26], wherein
the plurality of grooves include a third groove located adjacent to the first groove in the longitudinal direction, and
a maximum length between the first groove and the third groove in the longitudinal direction is shorter than a length of a pitch of the helical member.

One or more embodiments of the present invention also include the following catheter system.
[28] A catheter system comprising the puncture catheter according to any one of above [20] to [27] further comprising a sheath having an inner cavity into which the puncture needle and the first tube are inserted, wherein
when a length in a radial direction of the tubular member between the outer surface of the tubular member and a portion of the wire located outermost in the radial direction of the tubular member is denoted by H, a maximum outer diameter of the first tube is denoted by d, a maximum outer diameter of the tubular member is denoted by s, and an inner diameter of the sheath is denoted by D,
a relationship of D>d>s+2H is satisfied.

In the puncture needle according to one or more embodiments of the present invention, the helical member is disposed around the tubular member. Therefore, the puncture needle easily punctures a target tissue, is difficult to come out of the target tissue, and allows easy adjustment of a puncture depth thereof. The puncture catheter and the catheter system each including the puncture needle also have the same effects.

### BRIEF DESCRIPTION OF THE DRAWINGS

FIG. 1 is a side view showing an example of a puncture needle according to one or more embodiments of the present invention.

FIG. 2 is a cross-sectional view of the puncture needle shown in FIG. 1.

FIG. 3 is an end view of a cut section along a line III-III shown in FIG. 1.

FIG. 4 is an end view of a cut section showing a modification of the puncture needle shown in FIG. 3.

FIG. 5 is an end view of a cut section showing a modification of the puncture needle shown in FIG. 3.

FIG. 6 is an end view of a cut section showing a modification of the puncture needle shown in FIG. 3.

FIG. 7 is an end view of a cut section along a line VII-VII shown in FIG. 1.

FIG. 8 is a side view (partial cross-sectional view) showing an example of a puncture catheter including the puncture needle according to one or more embodiments of the present invention.

FIG. 9 is a side view showing an example of the puncture needle according to one or more embodiments of the present invention.

FIG. 10 is a cross-sectional view of the puncture needle shown in FIG. 9.

FIG. 11 is a side view showing a tubular member included in the puncture needle in FIG. 9.

FIG. 12 is a side view (partial cross-sectional view) showing an example of the puncture catheter including the puncture needle according to one or more embodiments of the present invention.

FIG. 13 is a side view showing an example of the puncture catheter including the puncture needle according to one or more embodiments of the present invention.

FIG. 14 is a cross-sectional view of the puncture catheter shown in FIG. 13.

FIG. 15 is a cross-sectional view showing a modification of the puncture catheter shown in FIG. 14.

FIG. 16 is a side view (partial cross-sectional view) showing an example of the puncture catheter including the puncture needle according to one or more embodiments of the present invention.

FIG. 17 is a side view showing an example of the puncture needle according to one or more embodiments of the present invention.

FIG. 18 is a cross-sectional view of the puncture needle shown in FIG. 17.

FIG. 19 is an end view of a cut section along a line XIX-XIX shown in FIG. 17.

FIG. 20 is an end view of a cut section showing a modification of the puncture needle shown in FIG. 19.

FIG. 21 is an end view of a cut section showing a modification of the puncture needle shown in FIG. 19.

FIG. 22 is an end view of a cut section showing a modification of the puncture needle shown in FIG. 19.

FIG. 23 is an end view of a cut section along a line XXIII-XXIII shown in FIG. 17.

FIG. 24 is a side view (partial cross-sectional view) showing an example of the puncture catheter including the puncture needle according to one or more embodiments of the present invention.

FIG. 25 is a side view (partial cross-sectional view) showing an example of a catheter system including the puncture catheter including the puncture needle according to one or more embodiments of the present invention.

FIG. 26 is a side view showing an example of the puncture needle and a first tube provided in the catheter system including the puncture catheter including the puncture needle according to one or more embodiments of the present invention.

FIG. 27 is a cross-sectional view of the puncture needle and the first tube shown in FIG. 26.

FIG. 28 is a cross-sectional view showing a modification of the puncture needle and the first tube shown in FIG. 27.

### DETAILED DESCRIPTION

One or more embodiments of the present invention will be specifically explained below based on the following embodiments, however, the present invention is not restricted by one or more embodiments described below of course, and can be certainly put into practice after appropriate modifications within in a range meeting the gist of the above and the below, all of which are included in the technical scope of the present invention. In the drawings, hatching, a reference sign for a member may be omitted for convenience, and in such a case, the description and other drawings should be referred to. In addition, sizes of various members in the drawings may differ from the actual sizes thereof, since priority is given to understanding the features of one or more embodiments of the present invention.

One or more embodiments of the puncture needle of the present invention have the gist of a medical puncture needle having a longitudinal direction, the puncture needle including: a tubular member having an inner cavity extending in the longitudinal direction of the puncture needle; and a helical member in which a wire is wound helically around the tubular member, wherein a distal end portion of the wire and the tubular member are not fixed to each other, or the distal end portion of the wire and the tubular member are not in contact with each other.

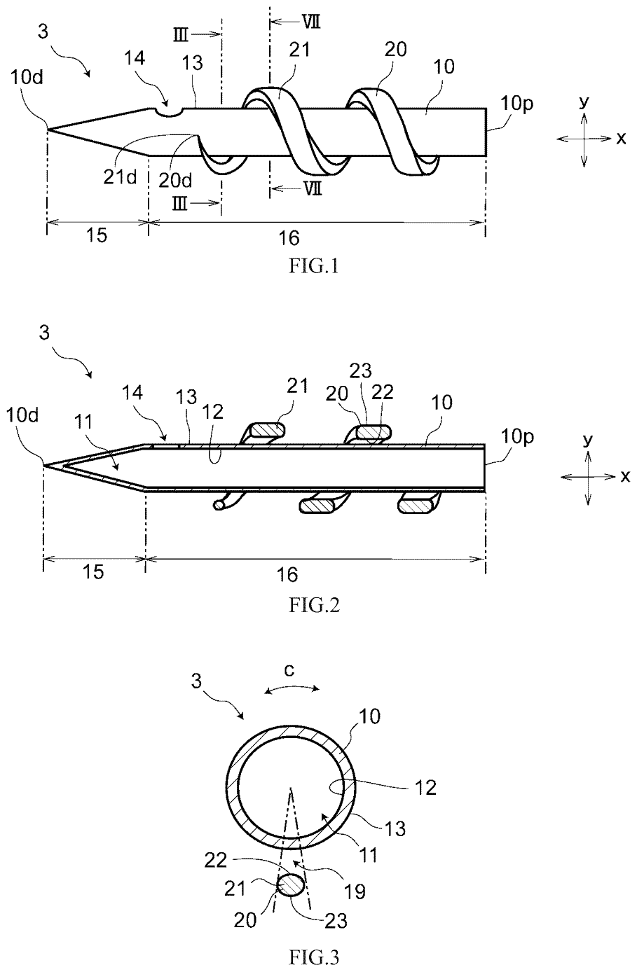

With reference to FIG. 1 to FIG. 28, the overall configurations of a puncture needle according to one or more embodiments of the present invention, a puncture catheter including the puncture needle, and a catheter system including the puncture catheter will be described.

FIG. 1 to FIG. 7 show a puncture needle 3 including a tubular member 10 and a helical member 20. FIG. 8 shows a puncture catheter 100 including the puncture needle 3, a tube 4, a sheath 5, and a handle 6.

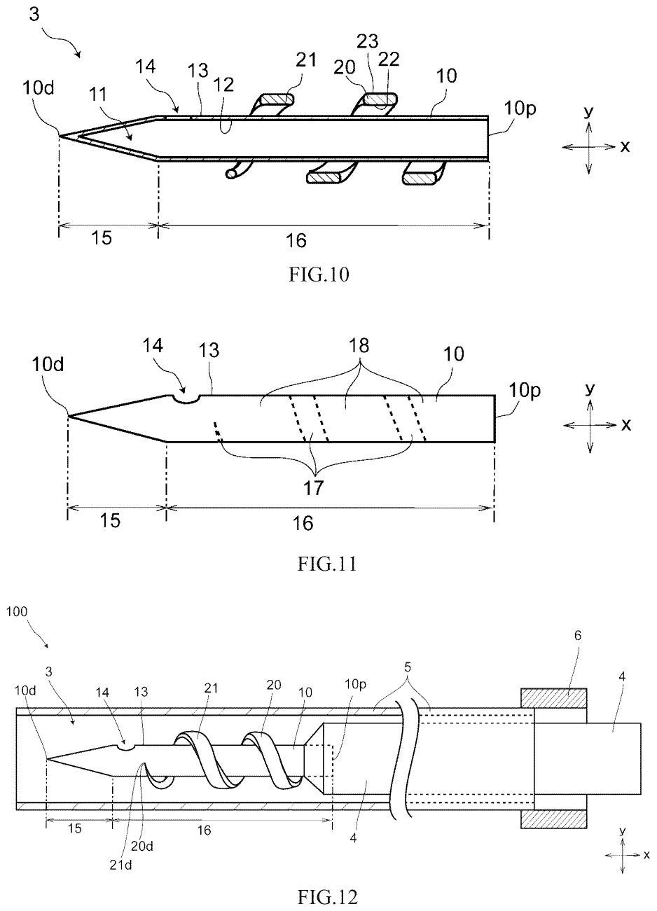

FIG. 9, FIG. 10, and FIG. 12 show the puncture needle 3 including the tubular member 10 and the helical member 20. FIG. 12 shows the puncture catheter 100 including the puncture needle 3, the tube 4, the sheath 5, and the handle 6.

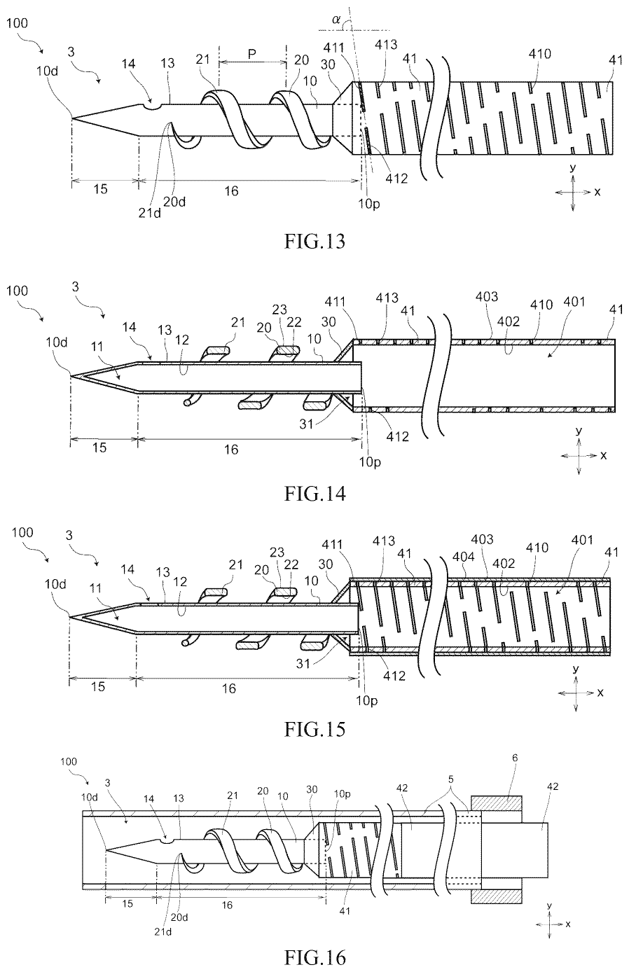

FIG. 13 to FIG. 15 show the puncture catheter 100 including the puncture needle 3 and a first tube 41. FIG. 16 shows the puncture catheter 100 including the puncture needle 3, the first tube 41, a second tube 42, the sheath 5, and the handle 6.

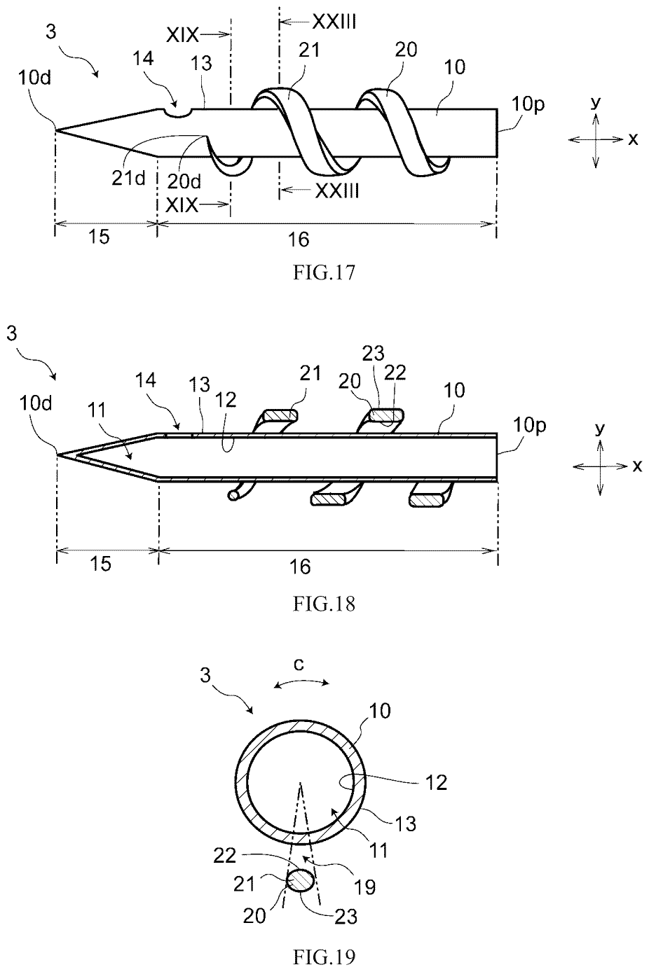

FIG. 17 to FIG. 23 show the puncture needle 3 including the tubular member 10 and the helical member 20. FIG. 24 shows the puncture catheter 100 including the puncture needle 3, the tube 4, the sheath 5, and the handle 6.

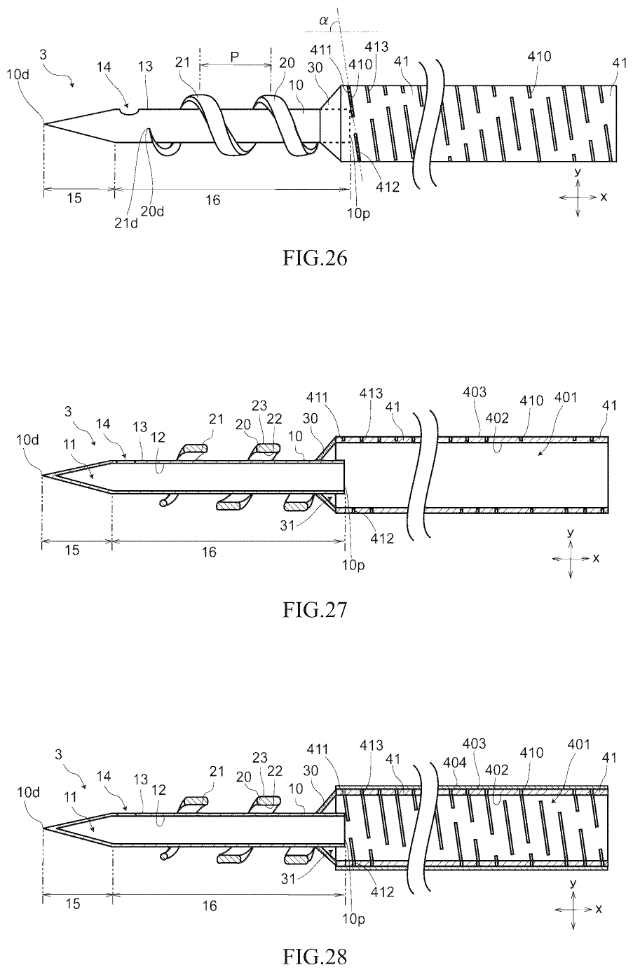

FIG. 25 shows the puncture catheter 100 including the puncture needle 3, the first tube 41, the sheath 5, and the handle 6, and a catheter system 200 including the puncture catheter 100. FIG. 26 to FIG. 28 show the puncture needle 3 and the first tube 41 included in the catheter system 200.

In these drawings, the longitudinal direction of the puncture needle 3 is indicated by x, the radial direction of the puncture needle 3 is indicated by y, and the circumferential direction of the puncture needle 3 is indicated by c. The radial direction y is a direction perpendicular to the longitudinal direction x. The longitudinal direction x of the puncture needle 3 can also be referred to as the extending direction of the puncture needle 3. For ease of understanding, these drawings illustrate one or more embodiments in which the longitudinal direction x of the puncture needle 3 coincides with the longitudinal direction of the tubular member 10, the radial direction y of the puncture needle 3 coincides with the radial direction of the tubular member 10, and the circumferential direction c of the puncture needle 3 coincides with the circumferential direction of the tubular member 10, but the present invention is not limited to these embodiments.

In this description, a proximal side refers to the hand side of a user with respect to the longitudinal direction x of the puncture needle 3, and a distal side refers to a side opposite to the proximal side, that is, a treatment target side. In addition, when each member is divided into two equal portions in the longitudinal direction x of the puncture needle 3, the portion of the member located on the distal side is referred to as distal portion, and the portion of the member located on the proximal side is referred to as proximal portion. The distal end of each member is an end located on the most distal side of the member. The proximal end of each member is an end located on the most proximal side of the member. An end portion of each member refers to a portion of the member including its end and an area therearound. That is, a distal end portion of each member refers to a portion of the member including its distal end and an area therearound, and a proximal end portion of each member refers to a portion of the member including its proximal end and an area therearound.

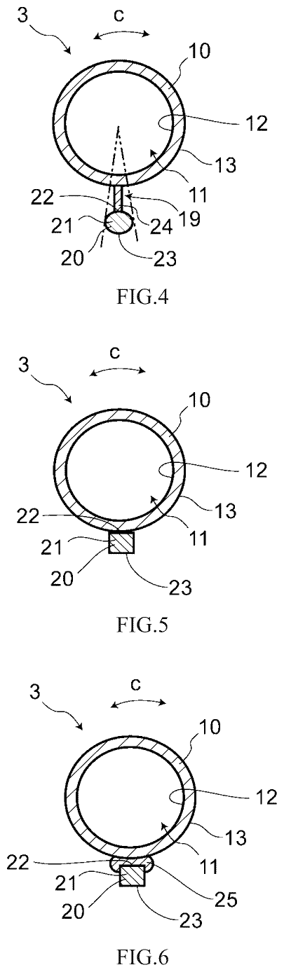

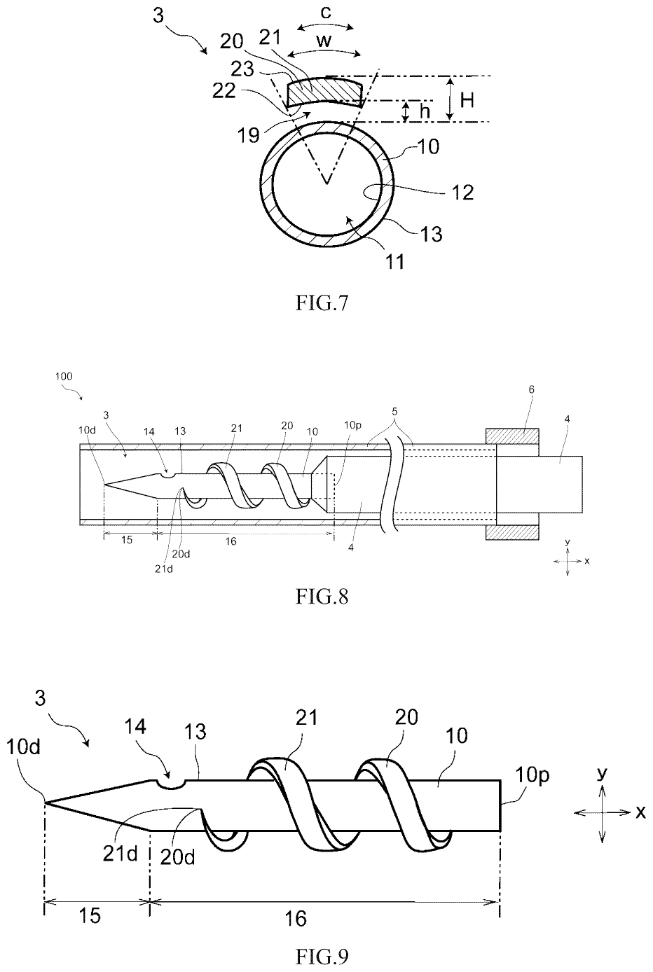

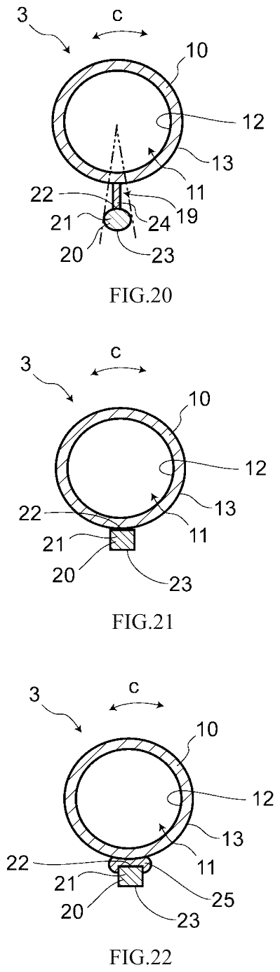

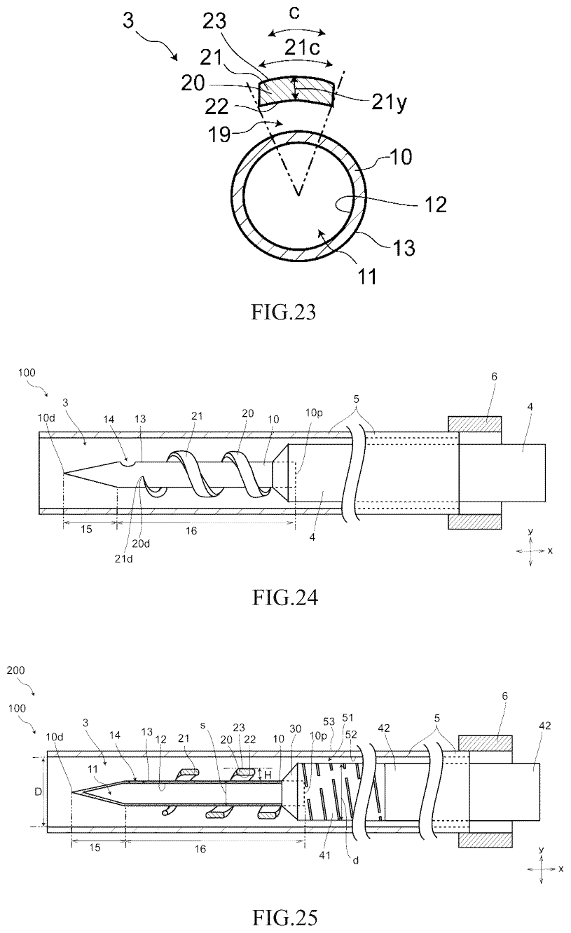

FIG. 1 is a side view showing an example of the puncture needle according to one or more embodiments of the present invention. FIG. 2 is a cross-sectional view of the puncture needle shown in FIG. 1. More specifically, FIG. 2 illustrates a cross-section passing through the central axis of the puncture needle and extending along the longitudinal direction of the puncture needle. FIG. 3 is an end view of a cut section along a line III-III shown in FIG. 1. FIG. 4 to FIG. 6 are each an end view of a cut section showing a modification of the puncture needle shown in FIG. 3. More specifically, FIG. 3 to FIG. 6 are each an end view of a cut section passing through a distal end portion of a wire. FIG. 7 is an end view of a cut section along a line VII-VII shown in FIG. 1. More specifically, FIG. 7 illustrates an end view of a cut section perpendicular to the longitudinal direction of the puncture needle and passing through the midpoint of the puncture needle in the longitudinal direction. FIG. 8 is a side view (partial cross-sectional view) showing an example of the puncture catheter including the puncture needle according to one or more embodiments of the present invention. FIG. 9 is a side view showing an example of the puncture needle according to one or more embodiments of the present invention. FIG. 10 is a cross-sectional view of the puncture needle shown in FIG. 9. More specifically, FIG. 10 illustrates a cross-section passing through the central axis of the puncture needle and extending along the longitudinal direction of the puncture needle. FIG. 11 is a side view showing a tubular member included in the puncture needle in FIG. 9. Here, the boundary between a region in which a wire is disposed radially outward of the tubular member and a region in which the wire is not disposed radially outward of the tubular member is indicated by a broken line. FIG. 12 is a side view (partial cross-sectional view) showing an example of the puncture catheter including the puncture needle according to one or more embodiments of the present invention. FIG. 13 is a side view showing an example of the puncture catheter including the puncture needle according to one or more embodiments of the present invention. FIG. 14 is a cross-sectional view of the puncture catheter shown in FIG. 13. FIG. 15 is a cross-sectional view showing a modification of the puncture catheter shown in FIG. 14. More specifically, FIG. 14 and FIG. 15 each illustrate a cross-section passing through the central axis of the puncture needle and extending along the longitudinal direction of the puncture needle. FIG. 16 is a side view (partial cross-sectional view) showing an example of the puncture catheter including the puncture needle according to one or more embodiments of the present invention. FIG. 17 is a side view showing an example of the puncture needle according to one or more embodiments of the present invention. FIG. 18 is a cross-sectional view of the puncture needle shown in FIG. 17. More specifically, FIG. 18 illustrates a cross-section passing through the central axis of the puncture needle and extending along the longitudinal direction of the puncture needle. FIG. 19 is an end view of a cut section along a line XIX-XIX shown in FIG. 17. FIG. 20 to FIG. 22 are each an end view of a cut section showing a modification of the puncture needle shown in FIG. 19. More specifically, FIG. 19 to FIG. 22 are each an end view of a cut section passing through a distal end portion of a wire. FIG. 23 is an end view of a cut section along a line XXIII-XXIII shown in FIG. 17. More specifically, FIG. 23 illustrates an end view of a cut section perpendicular to the longitudinal direction of the puncture needle and passing through the midpoint of the puncture needle in the longitudinal direction. FIG. 24 is a side view (partial cross-sectional view) showing an example of the puncture catheter including the puncture needle according to one or more embodiments of the present invention. FIG. 25 is a side view (partial cross-sectional view) showing an example of the catheter system including the puncture catheter including the puncture needle according to one or more embodiments of the present invention. FIG. 26 is a side view showing an example of the puncture needle and a first tube provided in the catheter system including the puncture catheter including the puncture needle according to one or more embodiments of the present invention. FIG. 27 is a cross-sectional view of the puncture needle and the first tube shown in FIG. 26. FIG. 28 is a cross-sectional view showing a modification of the puncture needle and the first tube shown in FIG. 27. More specifically, FIG. 27 and FIG. 28 each illustrate a cross-section passing through the central axis of the puncture needle and extending along the longitudinal direction of the puncture needle.

First, the puncture needle 3 according to one or more embodiments of the present invention will be described with reference to FIG. 1 to FIG. 28. The puncture needle 3 is for medical use and is intended to puncture a target tissue. As shown in FIG. 1, FIG. 2, FIG. 8 to FIG. 10, FIG. 12 to FIG. 18, and FIG. 24 to FIG. 28, the puncture needle 3 has the longitudinal direction x. The puncture needle 3 includes the tubular member 10 and the helical member 20.

As shown in FIG. 1, FIG. 2, FIG. 9, FIG. 10, FIG. 17, FIG. 18, FIG. 26, and FIG. 27, the tubular member 10 is a member having a tubular shape and has an inner cavity 11 extending in the longitudinal direction x of the puncture needle 3. A liquid such as a cell preparation or a drug solution can be delivered to the target tissue through the inner cavity 11.

As shown in FIG. 1, FIG. 2, FIG. 9, FIG. 10, FIG. 17, FIG. 18, FIG. 26, and FIG. 27, the puncture needle 3 includes the helical member 20 in which a wire 21 is wound helically around the tubular member 10.

The puncture needle 3 is configured such that, as shown in FIG. 3, FIG. 5, FIG. 19, and FIG. 21, a distal end portion of the wire 21 and the tubular member 10 are not fixed to each other, or, as shown in FIG. 3, FIG. 4, FIG. 6, FIG. 19, FIG. 20, and FIG. 22, the distal end portion of the wire 21 and the tubular member 10 are not in contact with each other. In other words, the puncture needle 3 has at least one of a configuration in which the distal end portion of the wire 21 and the tubular member 10 are not fixed to each other and a configuration in which the distal end portion of the wire 21 and the tubular member 10 are not in contact with each other. As shown in FIG. 5 and FIG. 21, the puncture needle 3 may have a configuration in which the distal end portion of the wire 21 and the tubular member 10 are in contact with each other but the distal end portion of the wire 21 and the tubular member 10 are not fixed to each other. As shown in FIG. 4, FIG. 6, FIG. 20, and FIG. 22, the puncture needle 3 may have a configuration in which the distal end portion of the wire 21 and the tubular member 10 are fixed to each other but the distal end portion of the wire 21 and the tubular member 10 are not in contact with each other. As shown in FIG. 3 and FIG. 19, in the puncture needle 3, the distal end portion of the wire 21 and the tubular member 10 do not have to be fixed to each other, and the distal end portion of the wire 21 and the tubular member 10 do not have to be in contact with each other.

The configuration in which the distal end portion of the wire 21 and the tubular member 10 are not fixed to each other refers to any configuration other than a configuration in which the distal end portion of the wire 21 and the tubular member 10 are fixed to each other. Examples of such a configuration include a configuration in which the distal end portion of the wire 21 and the tubular member 10 are not fixed via an adhesive or another member and the distal end portion of the wire 21 and the tubular member 10 are not fixed by welding.

The configuration in which the distal end portion of the wire 21 and the tubular member 10 are not in contact with each other refers to any configuration other than a configuration in which the distal end portion of the wire 21 and the tubular member 10 are in contact with each other. Examples of such a configuration include a configuration in which the inner surface of the distal end portion of the wire 21 and an outer surface 13 of the tubular member 10 are separated from each other via a gap or another member.

The distal end portion of the wire 21 refers to a portion of the wire 21 including its distal end 21d and an area therearound. The range of the distal end portion of the wire 21 may be, for example, the following range. The range of the distal end portion of the wire 21 may be a range from the distal end 21d of the wire 21 to a point located on the proximal side from the distal end 21 d of the wire 21 at a distance that is ⅓ of the length of the wire 21 when the wire 21 is divided into three equal portions in the longitudinal direction x of the puncture needle 3. The range of the distal end portion of the wire 21 may be a range from the distal end 21d of the wire 21 to a point located on the proximal side from the distal end 21d of the wire 21 at a distance that is ¼ of the length of the wire 21 when the wire 21 is divided into four equal portions in the longitudinal direction x of the puncture needle 3. The range of the distal end portion of the wire 21 may be a range from the distal end 21d of the wire 21 to a point located on the proximal side from the distal end 21d of the wire 21 at a distance that is ⅕ of the length of the wire 21 when the wire 21 is divided into five equal portions in the longitudinal direction x of the puncture needle 3.

Since the helical member 20 is disposed around the tubular member 10 in the puncture needle 3, the puncture needle 3 can be easily screwed into the target tissue by rotating the puncture needle 3 about the tubular member 10 as an axis. Accordingly, it becomes easier for the puncture needle 3 to puncture the target tissue. In addition, the wire 21 constituting the helical member 20 bites into the tissue, so that it becomes difficult for the puncture needle 3 to come out of the tissue. Furthermore, the puncture depth of the puncture needle 3 can be easily adjusted by adjusting the rotation of the puncture needle 3.

The puncture needle 3 is used when administering a liquid such as a cell preparation or a drug solution to the target tissue. Specifically, the puncture needle 3 can be used when directly administering such a liquid to an organ in the body such as the heart, kidneys, or liver. For example, the puncture needle 3 can be used when directly administering an iPS cell suspension to the liver or kidneys, or when directly administering a myocardial regenerative cell preparation to the heart, more specifically to the cardiac muscle.

It is preferable that the puncture needle 3 is intended to puncture organs in the body. The organs in the body refer to organs within the body, particularly those located in the abdominal or thoracic regions, and are portions commonly referred to as viscera.

The puncture needle 3 can be made of a metal or resin, for example. The entire puncture needle 3 may be made of only a metal or may be made of only a resin. A part of the puncture needle 3 may be made of a metal, and the other portion of the puncture needle 3 may be made of a resin. The tubular member 10 and the helical member 20 may be made of the same material or may be made of different materials.

It is preferable that the puncture needle 3 is made of only a metal. Examples of the metal forming the puncture needle 3 include stainless steels such as SUS304 and SUS316, platinum, nickel, cobalt, chromium, titanium, tungsten, gold, Ni—Ti alloys, Co—Cr alloys, and combinations thereof.

Examples of the resin forming the puncture needle 3 include polyether ether ketone (PEEK) and polycarbonate (PC). When the puncture needle 3 is made of only a resin without using any metal, the puncture needle 3 can be used for patients with metal allergies.

The length of the puncture needle 3 in the longitudinal direction x of the puncture needle 3 can be set to 2 mm or more, 3 mm or more, 4 mm or more, etc. The length of the puncture needle 3 in the longitudinal direction x of the puncture needle 3 can be set to 50 mm or less, 30 mm or less, 10 mm or less, etc. The length of the puncture needle 3 in the longitudinal direction x of the puncture needle 3 refers to the maximum length among lengths of the puncture needle 3 in the longitudinal direction x of the puncture needle 3. When the puncture needle 3 is used as a puncture needle for puncturing the cardiac muscle, it is preferable that the length of the puncture needle 3 in the longitudinal direction x of the puncture needle 3 is 5 mm.

The length of the puncture needle 3 in the radial direction y of the puncture needle 3 can be set to 0.2 mm or more, 0.3 mm or more, 0.4 mm or more, etc. The length of the puncture needle 3 in the radial direction y of the puncture needle 3 can be set to 10 mm or less, 5 mm or less, 1 mm or less, etc. The length of the puncture needle 3 in the radial direction y of the puncture needle 3 refers to the maximum length among lengths of the puncture needle 3 in the radial direction y of the puncture needle 3. When the puncture needle 3 is used as a puncture needle for puncturing the cardiac muscle, it is preferable that the length of the puncture needle 3 in the radial direction y of the puncture needle 3 is 0.45 mm.

The tubular member 10 can be configured to have an inner surface 12 facing the inner cavity 11 and the outer surface 13 facing the exterior thereof. The tubular member 10 has a distal end 10d and a proximal end 10p in the longitudinal direction x of the puncture needle 3. As for the tubular member 10, it is preferable that the distal end 10d is closed and the proximal end 10p is open.

As shown in FIG. 1, FIG. 2, FIG. 8 to FIG. 18, and FIG. 24 to FIG. 28, it is preferable that the tubular member 10 includes: a tapered portion 15 having an outer diameter decreasing toward the distal side; and a straight tube portion 16 located on the proximal side with respect to the tapered portion 15. In particular, it is preferable that the tapered portion 15 is provided at a portion including the distal end 10d of the tubular member 10. Accordingly, it is made possible for a distal end portion of the tubular member 10 to puncture the target tissue. In addition, when the puncture needle 3 is rotated in the circumferential direction c of the puncture needle 3, the deviation of the rotation axis thereof is reduced, thereby making it easier to improve stability during puncturing with the puncture needle 3.

As shown in FIG. 1, FIG. 2, FIG. 8 to FIG. 18, and FIG. 24 to FIG. 28, it is preferable that a hole 14 providing communication between the inner cavity 11 of the tubular member 10 and the exterior of the puncture needle 3 is formed in the tubular member 10. A liquid such as a cell preparation or a drug solution delivered through the inner cavity 11 is delivered to the target tissue through the hole 14.

As shown in FIG. 1, FIG. 2, FIG. 8 to FIG. 18, and FIG. 24 to FIG. 28, it is preferable that the hole 14 is formed in the straight tube portion 16. Accordingly, it becomes easier for a liquid such as a cell preparation or a drug solution delivered through the inner cavity 11 to be discharged to the radially outer side of the tubular member 10.

It is preferable that the hole 14 is a hole through which the liquid can be discharged from the inner cavity 11 of the tubular member 10 toward the radially outer side of the tubular member 10. Therefore, it is preferable that the hole 14 is located on the proximal side with respect to the distal end 10d of the tubular member 10 and on the distal side with respect to the proximal end 10p of the tubular member 10.

Only one hole 14 may be formed in the tubular member 10, or a plurality of holes 14 may be formed in the tubular member 10. When only one hole 14 is formed in the tubular member 10, it becomes easier to administer a liquid such as a cell preparation or a drug solution in a pinpoint manner. When a plurality of holes 14 are formed in the tubular member 10, it becomes easier to administer a liquid such as a cell preparation or a drug solution over a wide area.

The outer shape of the hole 14 when observed in a direction perpendicular to the longitudinal direction x of the puncture needle 3 can be a polygonal shape such as a triangular shape, a quadrilateral shape, or a pentagonal shape, a circular shape, an elliptical shape, or a combination thereof. The polygonal shape includes not only a shape in which the vertex of each corner portion is clearly defined and each side is straight but also a polygonal shape with curved corners and a shape in which at least one or some of the sides are curved.

As shown in FIG. 9 and FIG. 11, it is preferable that the tubular member 10 has a wire-arrangement region 17 in which the wire 21 is disposed radially outward of the tubular member 10 and a wire-non-arrangement region 18 in which the wire 21 is not disposed radially outward of the tubular member 10. It is also preferable that the hole 14 is formed in the wire-non-arrangement region 18 of the tubular member 10. When the hole 14 providing communication between the inner cavity 11 of the tubular member 10 and the exterior of the puncture needle 3 is formed in the wire-non-arrangement region 18, the liquid discharged from the hole 14 can be inhibited from colliding with the wire 21 and thus moving in an unintended direction. From the viewpoint of further enhancing the effect of inhibiting the liquid discharged from the hole 14 from colliding with the wire 21 and thus moving in an unintended direction, it is preferable that the hole 14 providing communication between the inner cavity 11 of the tubular member 10 and the exterior of the puncture needle 3 is not formed in the wire-arrangement region 17 of the tubular member 10.

The helical member 20 is formed by helically winding the wire 21. When the wire 21 is wound, the wire 21 has an inner surface located on the inner side thereof and an outer surface located on the outer side thereof. The inner cavity formed on the inner surface side of the wire 21 is the inner cavity of the helical member 20. The tubular member 10 is disposed in the inner cavity of the helical member 20. The helical member 20 can be configured to have an inner surface 22 facing the inner cavity of the helical member 20 and an outer surface 23 facing the exterior thereof. The helical member 20 has a distal end 20d and a proximal end in the longitudinal direction x of the puncture needle 3.

Only one helical member 20 may be provided in the puncture needle 3, or a plurality of helical members 20 may be provided in the puncture needle 3.

The cross-sectional shape of the wire 21 in a cross-section perpendicular to the longitudinal direction x of the puncture needle 3 can be a polygonal shape such as a triangular shape, a quadrilateral shape, or a pentagonal shape, a circular shape, an elliptical shape, or a combination thereof. In the cross-section perpendicular to the longitudinal direction x of the puncture needle 3, the cross-sectional shape of the wire 21 may be a polygonal shape or a quadrilateral shape. The cross-sectional shape of the wire 21 in the cross-section perpendicular to the longitudinal direction x of the puncture needle 3 may be the same from the distal end 21d to the proximal end of the wire 21. When the cross-sectional shape of the wire 21 in the cross-section perpendicular to the longitudinal direction x of the puncture needle 3 is the same from the distal end 21d to the proximal end of the wire 21, the size of the cross-sectional shape may be the same (congruent) or different (similar) depending on the position in the longitudinal direction x of the puncture needle 3.

The cross-sectional shape of the wire 21 in the cross-section perpendicular to the longitudinal direction x of the puncture needle 3 may be different depending on the position in the longitudinal direction x of the puncture needle 3. For example, as shown in FIG. 1, FIG. 7, FIG. 17, and FIG. 23, the cross-sectional shape of the wire 21 in a cross-section perpendicular to the longitudinal direction x of the puncture needle 3 and passing through the midpoint of the puncture needle 3 in the longitudinal direction x is a rectangular shape, and as shown in FIG. 1, FIG. 3, FIG. 17, and FIG. 19, the cross-sectional shape of the wire 21 in a cross-section perpendicular to the longitudinal direction x of the puncture needle 3 and passing through the distal end portion of the wire 21 may be an elliptical shape. Alternatively, as shown in FIG. 7 and FIG. 23, the cross-sectional shape of the wire 21 in the cross-section perpendicular to the longitudinal direction x of the puncture needle 3 and passing through the midpoint of the puncture needle 3 in the longitudinal direction x may be a rectangular shape in which two long sides have an arc shape.

As shown in FIG. 2 to FIG. 7, FIG. 10, FIG. 14, FIG. 15, FIG. 18 to FIG. 23, FIG. 25, FIG. 27, and FIG. 28, it is preferable that the wire 21 has a solid structure. The entire wire 21 may have a solid structure, or only a part of the wire 21 may have a solid structure. In particular, it is preferable that the entire distal end portion of the wire 21 has a solid structure. When the wire 21 is made to have a solid structure, it becomes easier to reduce the diameter of the wire 21. However, although not illustrated, one or more embodiments in which the wire 21 has a hollow structure is also permissible. For example, the wire 21 may have an inner cavity formed to extend along the central axis of the wire 21.

The wire 21 may be a single wire or a stranded wire.

As shown in FIG. 1, FIG. 8, FIG. 9, FIG. 12, FIG. 13, FIG. 16, FIG. 17, FIG. 24, and FIG. 26, it is preferable that the distal end portion of the wire 21 has a tapered shape. When the wire 21 has a tapered shape, it can be made easier to screw the wire 21 into the tissue.

It is preferable that the distal end 21d of the wire 21 is located on the proximal side with respect to the distal end 10d of the tubular member 10. The distal end 21d of the wire 21 may be located on the distal side with respect to the proximal end of the tapered portion 15 of the tubular member 10, but it is preferable that the distal end 21d of the wire 21 is located on the proximal side with respect to the proximal end of the tapered portion 15 of the tubular member 10. The wire 21 may be disposed only radially outward of the straight tube portion 16 of the tubular member 10 and may not necessarily be disposed radially outward of the tapered portion 15 of the tubular member 10. Owing to this configuration, the distal end portion of the tubular member 10 can be brought into contact with the target tissue before a distal end portion of the helical member 20. Accordingly, it can be made easier to rotate the puncture needle 3 about the tubular member 10 as an axis when rotating the puncture needle 3 in the circumferential direction c of the puncture needle 3, so that the deviation of the rotation axis thereof is easily reduced, thereby making it easier to improve stability during puncturing with the puncture needle 3.

It is preferable that, in a section where the distal end portion of the wire 21 is located in the longitudinal direction x of the puncture needle 3, the outer surface 13 of the tubular member 10 and the inner surface 22 of the helical member 20 are separated from each other. It is preferable that, in a section where a distal portion of the helical member 20 is located in the longitudinal direction x of the puncture needle 3, the outer surface 13 of the tubular member 10 and the inner surface 22 of the helical member 20 are separated from each other as shown in FIG. 3, FIG. 4, FIG. 6, FIG. 19, FIG. 20, and FIG. 22. Owing to this configuration, it becomes easier for the tissue to penetrate into the space between the tubular member 10 and the wire 21. Accordingly, after the puncture needle 3 has punctured the target tissue, it becomes difficult for the puncture needle 3 to come out of the tissue.

As shown in FIG. 3, FIG. 4, FIG. 6, FIG. 19, FIG. 20, and FIG. 22, it is preferable that, in a section where the tapered shape of the wire 21 is located in the longitudinal direction x of the puncture needle 3, the outer surface 13 of the tubular member 10 and the inner surface 22 of the helical member 20 are separated from each other. Owing to this configuration, it becomes easier for the tissue to penetrate into the space between the tubular member 10 and the wire 21. Accordingly, after the puncture needle 3 has punctured the target tissue, it becomes difficult for the puncture needle 3 to come out of the tissue.

It is preferable that, in a section where the distal portion of the helical member 20 is located in the longitudinal direction x of the puncture needle 3, the outer surface 13 of the tubular member 10 and the inner surface 22 of the helical member 20 are separated from each other due to the presence of a gap in a space 19 between the outer surface 13 of the tubular member 10 and the inner surface 22 of the helical member 20 as shown in FIG. 3, FIG. 4, FIG. 19, and FIG. 20. In the section where the distal portion of the helical member 20 is located in the longitudinal direction x of the puncture needle 3, in addition to the gap, a member may be disposed in the space 19 between the outer surface 13 of the tubular member 10 and the inner surface 22 of the helical member 20 as shown in FIG. 4 and FIG. 20. It is preferable that, in the section where the distal portion of the helical member 20 is located in the longitudinal direction x of the puncture needle 3, only a gap is present in the space 19 between the outer surface 13 of the tubular member 10 and the inner surface 22 of the helical member 20 as shown in FIG. 3 and FIG. 19. Owing to this configuration, it becomes easier for the tissue to penetrate into the space between the tubular member 10 and the wire 21. Accordingly, after the puncture needle 3 has punctured the target tissue, it becomes difficult for the puncture needle 3 to come out of the tissue.

It is preferable that, in the section where the tapered shape of the wire 21 is located in the longitudinal direction x of the puncture needle 3, the outer surface 13 of the tubular member 10 and the inner surface 22 of the helical member 20 are separated from each other due to the presence of a gap in the space 19 between the outer surface 13 of the tubular member 10 and the inner surface 22 of the helical member 20 as shown in FIG. 3, FIG. 4, FIG. 19, and FIG. 20. In the section where the tapered shape of the wire 21 is located in the longitudinal direction x of the puncture needle 3, in addition to the gap, a member may be disposed in the space 19 between the outer surface 13 of the tubular member 10 and the inner surface 22 of the helical member 20 as shown in FIG. 4 and FIG. 20. It is preferable that, in the section where the tapered shape of the wire 21 is located in the longitudinal direction x of the puncture needle 3, only a gap is present in the space 19 between the outer surface 13 of the tubular member 10 and the inner surface 22 of the helical member 20 as shown in FIG. 3 and FIG. 19. Owing to this configuration, it becomes easier for the tissue to penetrate into the space between the tubular member 10 and the wire 21. Accordingly, after the puncture needle 3 has punctured the target tissue, it becomes difficult for the puncture needle 3 to come out of the tissue.

The space 19 between the outer surface 13 of the tubular member 10 and the inner surface 22 of the helical member 20 is a portion located between the outer surface 13 of the tubular member 10 and the inner surface 22 of the helical member 20 in the radial direction of the tubular member 10. In FIG. 3, FIG. 4, FIG. 7, FIG. 19, FIG. 20, and FIG. 23, rays extending in the radial direction of the tubular member 10 from the central axis of the tubular member 10 are indicated by an alternate long and two short dashes line.

As shown in FIG. 4 and FIG. 20, the puncture needle 3 may include a connection member 24.

More specifically, in the section where the distal portion of the helical member 20 is located in the longitudinal direction x of the puncture needle 3, the outer surface 13 of the tubular member 10 and the inner surface 22 of the helical member 20 may be connected via the connection member 24. Also, in the section where the distal end portion of the wire 21 is located in the longitudinal direction x of the puncture needle 3, the outer surface 13 of the tubular member 10 and the inner surface 22 of the helical member 20 may be connected via the connection member 24. It is preferable that the length of the connection member 24 in the circumferential direction c of the puncture needle 3 is shorter than the length of the wire 21 in the circumferential direction c of the puncture needle 3. Furthermore, it is preferable that, in addition to the connection member 24, a gap is present in the space 19 between the outer surface 13 of the tubular member 10 and the inner surface 22 of the helical member 20. Owing to this configuration, it becomes easier for the tissue to penetrate into the space between the tubular member 10 and the wire 21. Accordingly, after the puncture needle 3 has punctured the target tissue, it becomes difficult for the puncture needle 3 to come out of the tissue. The length of the connection member 24 in the circumferential direction c of the puncture needle 3 refers to the maximum length among lengths of the connection member 24 in the circumferential direction c of the puncture needle 3. The length of the wire 21 in the circumferential direction c of the puncture needle 3 refers to the maximum length among lengths of the wire 21 in the circumferential direction c of the puncture needle 3.

In the section where the tapered shape of the wire 21 is located in the longitudinal direction x of the puncture needle 3, the outer surface 13 of the tubular member 10 and the inner surface 22 of the helical member 20 may be connected via the connection member 24. In this case, it is preferable that the length of the connection member 24 in the circumferential direction c of the puncture needle 3 is shorter than the length of the wire 21 in the circumferential direction c of the puncture needle 3. Furthermore, it is preferable that, in addition to the connection member 24, a gap is present in the space 19 between the outer surface 13 of the tubular member 10 and the inner surface 22 of the helical member 20. Owing to this configuration, it becomes easier for the tissue to penetrate into the space between the tubular member 10 and the wire 21. Accordingly, after the puncture needle 3 has punctured the target tissue, it becomes difficult for the puncture needle 3 to come out of the tissue. The length of the connection member 24 in the circumferential direction c of the puncture needle 3 refers to the maximum length among lengths of the connection member 24 in the circumferential direction c of the puncture needle 3. The length of the wire 21 in the circumferential direction c of the puncture needle 3 refers to the maximum length among lengths of the wire 21 in the circumferential direction c of the puncture needle 3.

The connection member 24 is, for example, an elongated, linear, or strip-like member, and may be provided along the wire 21. In this case, it is preferable that the length of the connection member 24 in the axial direction of the connection member 24 is shorter than the length of the wire 21 in the axial direction of the wire 21. In the longitudinal direction x of the puncture needle 3, the connection member 24 may be provided only in the section where the distal portion of the wire 21 is located, or may be provided only in the section where the distal end portion of the wire 21 is located. The tubular member 10, the helical member 20, and the connection member 24 may be made of the same material, or may be made of different materials.

In the section where the distal portion of the helical member 20 is located in the longitudinal direction x of the puncture needle 3, as shown in FIG. 5 and FIG. 21, the inner surface 22 of the helical member 20 and the outer surface 13 of the tubular member 10 may be in contact with each other, but the inner surface 22 of the helical member 20 and the outer surface 13 of the tubular member 10 may not necessarily be fixed to each other. A configuration in which the inner surface 22 of the helical member 20 and the outer surface 13 of the tubular member 10 are not fixed to each other refers to, for example, a configuration in which the inner surface 22 of the helical member 20 and the outer surface 13 of the tubular member 10 are not bonded together by an adhesive and the inner surface 22 of the helical member 20 and the outer surface 13 of the tubular member 10 are not fixed by welding.

As shown in FIG. 5 and FIG. 21, the inner surface 22 of the distal end portion of the helical member 20 and the outer surface 13 of the tubular member 10 may be in contact with each other. In a state where the inner surface 22 of the helical member 20 and the outer surface 13 of the tubular member 10 are in contact with each other, the inner surface 22 of the helical member 20 and the outer surface 13 of the tubular member 10 may not necessarily be fixed to each other. A configuration in which the inner surface 22 of the helical member 20 and the outer surface 13 of the tubular member 10 are not fixed to each other refers to any configuration other than a configuration in which the inner surface 22 of the helical member 20 and the outer surface 13 of the tubular member 10 are fixed to each other, and, for example, refers to a configuration in which the inner surface 22 of the helical member 20 and the outer surface 13 of the tubular member 10 are not fixed via an adhesive or another member and the inner surface 22 of the helical member 20 and the outer surface 13 of the tubular member 10 are not fixed by welding. In the section where the tapered shape of the wire 21 is located in the longitudinal direction x of the puncture needle 3, as shown in FIG. 5 and FIG. 21, the inner surface 22 of the helical member 20 and the outer surface 13 of the tubular member 10 are in contact with each other, but the inner surface 22 of the helical member 20 and the outer surface 13 of the tubular member 10 may not necessarily be fixed to each other.

The distal end portion of the helical member 20 refers to a portion of the helical member 20 including the distal end 20d and an area therearound. The range of the distal end portion of the helical member 20 may be, for example, the following range. The range of the distal end portion of the helical member 20 may be a range from the distal end 20d of the helical member 20 to a point located on the proximal side from the distal end 20d of the helical member 20 at a distance that is ⅓ of the length of the helical member 20 when the helical member 20 is divided into three equal portions in the longitudinal direction x of the puncture needle 3. The range of the distal end portion of the helical member 20 may be a range from the distal end 20d of the helical member 20 to a point located on the proximal side from the distal end 20 d of the helical member 20 at a distance that is ¼ of the length of the helical member 20 when the helical member 20 is divided into four equal portions in the longitudinal direction x of the puncture needle 3. The range of the distal end portion of the helical member 20 may be a range from the distal end 20d of the helical member 20 to a point located on the proximal side from the distal end 20 d of the helical member 20 at a distance that is ⅕ of the length of the helical member 20 when the helical member 20 is divided into five equal portions in the longitudinal direction x of the puncture needle 3.

In the section where the distal portion of the helical member 20 is located in the longitudinal direction x of the puncture needle 3, as shown in FIG. 6 and FIG. 22, the inner surface 22 of the helical member 20 and the outer surface 13 of the tubular member 10 may not necessarily be in contact with each other, but the inner surface 22 of the helical member 20 and the outer surface 13 of the tubular member 10 may be fixed by an adhesive 25.

The inner surface 22 of the distal end portion of the helical member 20 and the outer surface 13 of the tubular member 10 may be fixed by adhesion. When fixed by adhesion, the inner surface 22 of the distal end portion of the helical member 20 and the outer surface 13 of the tubular member 10 can be bonded by the adhesive 25 as shown in FIG. 6 and FIG. 22. The inner surface 22 of the distal end portion of the helical member 20 and the outer surface 13 of the tubular member 10 may not necessarily be in contact with each other. In the section where the tapered shape of the wire 21 is located in the longitudinal direction x of the puncture needle 3, as shown in FIG. 6 and FIG. 22, the inner surface 22 of the helical member 20 and the outer surface 13 of the tubular member 10 may not necessarily be in contact with each other, but the inner surface 22 of the helical member 20 and the outer surface 13 of the tubular member 10 may be fixed by the adhesive 25.

As the adhesive 25, for example, a polyurethane-based, epoxy-based, cyano-based, fluorine-based, or silicone-based adhesive can be used.

As shown in FIG. 7, in the cross-section perpendicular to the longitudinal direction x of the puncture needle 3 and passing through the midpoint of the puncture needle 3 in the longitudinal direction x, when the length in the radial direction of the tubular member 10 between the outer surface 13 of the tubular member 10 and a portion of the wire 21 located outermost in the radial direction of the tubular member 10 is denoted by H, the separation distance of the space 19 between the outer surface 13 of the tubular member 10 and the inner surface 22 of the helical member 20 is denoted by h, and the width of the wire 21 in the circumferential direction c of the puncture needle 3 is denoted by W, it is preferable that the puncture needle 3 satisfies relationships of 2h<H and H−h<W. Owing to this configuration, it becomes easier for the tissue to penetrate into the space between the tubular member 10 and the wire 21. Accordingly, after the puncture needle 3 has punctured the target tissue, it becomes difficult for the puncture needle 3 to come out of the tissue.

It is preferable that the distal end 20d of the helical member 20 is located on the proximal side with respect to the distal end 10d of the tubular member 10. The distal end 20d of the helical member 20 may be located on the distal side with respect to the proximal end of the tapered portion 15 of the tubular member 10, but it is preferable that the distal end 20d of the helical member 20 is located on the proximal side with respect to the proximal end of the tapered portion 15 of the tubular member 10. The wire 21 may be disposed only radially outward of the straight tube portion 16 of the tubular member 10 and may not necessarily be disposed radially outward of the tapered portion 15 of the tubular member 10. Owing to this configuration, the distal end portion of the tubular member 10 can be brought into contact with the target tissue before the distal end portion of the helical member 20. Accordingly, it can be made easier to rotate the puncture needle 3 about the tubular member 10 as an axis when rotating the puncture needle 3 in the circumferential direction c of the puncture needle 3, so that the deviation of the rotation axis thereof is easily reduced, thereby making it easier to improve stability during puncturing with the puncture needle 3.

A part of the tubular member 10 and a part of the helical member 20 may be fixed to each other. More specifically, it is preferable that a proximal portion of the helical member 20 is fixed to a proximal portion of the tubular member 10, and it is preferable that a proximal end portion of the helical member 20 is fixed to the proximal portion of the tubular member 10. Accordingly, it becomes easier to stably screw the puncture needle 3 into the tissue without the position of the helical member 20 being changed relative to the tubular member 10. The tubular member 10 and the helical member 20 may be indirectly fixed via another member, but it is preferable that a part of the tubular member 10 and a part of the helical member 20 are directly fixed to each other without another member being interposed therebetween.

Direct fixation of the tubular member 10 and the helical member 20 refers to a state where the tubular member 10 and the helical member 20 are fixed to each other without another member being interposed therebetween. For example, fixation of the tubular member 10 and the helical member 20 by the adhesive 25 as described above, fixation of the tubular member 10 and the helical member 20 by welding, etc., correspond to the direct fixation.

Indirect fixation of the tubular member 10 and the helical member 20 refers to a state where the tubular member 10 and the helical member 20 are fixed via another member. For example, fixation of the tubular member 10 and the helical member 20 via the connection member 24, which connects the outer surface 13 of the tubular member 10 and the inner surface 22 of the helical member 20, as described above, corresponds to the indirect fixation.

The proximal end portion of the helical member 20 refers to a portion of the helical member 20 including its proximal end and an area therearound. The range of the proximal end portion of the helical member 20 may be, for example, the following range. The range of the proximal end portion of the helical member 20 may be a range from the proximal end of the helical member 20 to a point located on the distal side from the proximal end of the helical member 20 at a distance that is ⅓ of the length of the helical member 20 when the helical member 20 is divided into three equal portions in the longitudinal direction x of the puncture needle 3. The range of the proximal end portion of the helical member 20 may be a range from the proximal end of the helical member 20 to a point located on the distal side from the proximal end of the helical member 20 at a distance that is ¼ of the length of the helical member 20 when the helical member 20 is divided into four equal portions in the longitudinal direction x of the puncture needle 3. The range of the proximal end portion of the helical member 20 may be a range from the proximal end of the helical member 20 to a point located on the distal side from the proximal end of the helical member 20 at a distance that is ⅕ of the length of the helical member 20 when the helical member 20 is divided into five equal portions in the longitudinal direction x of the puncture needle 3.

The inner diameter of the helical member 20 may be four times or less or three times or less the outer diameter of the tubular member 10, but may be twice or less the outer diameter of the tubular member 10. By adopting this configuration, the gap between the tubular member 10 and the helical member 20 tends to be smaller, thereby making it easier to screw the puncture needle 3 into the target tissue by rotating the puncture needle 3 about the tubular member 10 as an axis. It is preferable that the inner diameter of the helical member 20 is larger than the outer diameter of the tubular member 10, and the inner diameter of the helical member 20 can be set to 1.1 times or more, 1.2 times or more, 1.3 times or more, etc., the outer diameter of the tubular member 10.

As shown in FIG. 23, it is preferable that, in the cross-section perpendicular to the longitudinal direction x of the puncture needle 3 and passing through the midpoint of the puncture needle 3 in the longitudinal direction x, a length 21y of the wire 21 in the radial direction of the tubular member 10 is shorter than a length 21c of the wire 21 in the circumferential direction of the tubular member 10. The longer the length 21y of the wire 21 in the radial direction of the tubular member 10 is, the easier it becomes to adjust the puncture depth; however, in some cases, it may become more difficult for the puncture needle 3 to puncture the tissue, requiring more time for puncture. By adopting the above configuration, the length 21y of the wire 21 in the radial direction of the tubular member 10 can be made relatively short, so that it becomes easier to adjust the puncture depth without making it excessively difficult for the puncture needle 3 to puncture the tissue. The length 21y of the wire 21 in the radial direction of the tubular member 10 refers to the maximum length among lengths of the wire 21 in the radial direction of the tubular member 10. The length 21c of the wire 21 in the circumferential direction of the tubular member 10 refers to the maximum length among lengths of the wire 21 in the circumferential direction of the tubular member 10.

As shown in FIG. 1 and FIG. 26, it is preferable that the tapered shape extend from the distal end 21d of the wire 21 to a position away from the distal end 21d of the wire 21 toward the proximal side by a length equal to ½ of a pitch P of the helical member 20. By having a tapered shape extending to the above position, it becomes easier for the wire 21 to enter the target tissue, so that it becomes easier to screw the puncture needle 3 into the target tissue. The tapered shape may be a shape that becomes narrower toward the distal end 21d of the wire 21.

It is preferable that the wire 21 has a section where a value obtained by dividing the length 21c of the wire 21 in the circumferential direction of the tubular member 10, measured in the cross-section perpendicular to the longitudinal direction x of the puncture needle 3, by the length 21y of the wire 21 in the radial direction of the tubular member 10, measured in the cross-section, decreases toward the distal side. Accordingly, the effect of making it easier to screw the wire 21 can be enhanced.

As shown in FIG. 26, it is preferable that the pitch P of the helical member 20 is larger than the inner diameter of the hole 14. Accordingly, it can be made easier to form the hole 14 even when forming the hole 14 in the wire-non-arrangement region 18 that is present between the wire-arrangement region 17 and the wire-arrangement region 17.

As shown in FIG. 26, it is preferable that the hole 14 is located on the distal side with respect to the distal end 21d of the wire 21. Accordingly, it can be made easier to form the hole 14 in the wire-non-arrangement region 18 even when the pitch P of the helical member 20 is relatively short.

Next, the puncture catheter 100 including the puncture needle 3 according to one or more embodiments of the present invention will be described with reference to FIG. 1 to FIG. 28. The components already described above are denoted by the same reference characters, and the description thereof is sometimes omitted.

As shown in FIG. 8, FIG. 12 to FIG. 16, FIG. 24, and FIG. 25, it is preferable that the puncture catheter 100 includes the puncture needle 3 having any of the above-described configurations.

As shown in FIG. 8, FIG. 12, and FIG. 24, the puncture catheter 100 may include the tube 4 connected to a proximal end portion of the puncture needle 3 and having an inner cavity extending in the longitudinal direction x of the puncture needle 3. It is preferable that the proximal end portion of the puncture needle 3 is connected to a distal end portion of the tube 4.

The tube 4 can be made of, for example, any of the materials exemplified as those that can form the puncture needle 3.

The tube 4 may be composed of only one member having a tubular shape, or may be composed of a plurality of members having a tubular shape.

As shown in FIG. 8, FIG. 12, and FIG. 24, the puncture catheter 100 may include the sheath 5 having an inner cavity into which the puncture needle 3 can be inserted. The tube 4 may also be insertable into the inner cavity of the sheath 5. It is preferable that the puncture needle 3 and the tube 4 are movable in the longitudinal direction x of the puncture needle 3 within the inner cavity of the sheath 5.

Since the sheath 5 is inserted into the body, the sheath 5 may have flexibility. Accordingly, the sheath 5 can be deformed along the shape of the body cavity. In addition, for shape retention, it is preferable that the sheath 5 has elasticity.

Examples of the sheath 5 include: a hollow body formed by arranging one or more wires in a predetermined pattern; a body obtained by coating at least one of the inner and outer surfaces of the hollow body with a resin; a resin tube; and combinations of these such as these connected in the longitudinal direction. Examples of a hollow body with a wire(s) arranged in a predetermined pattern include a tubular body having a mesh structure formed by wires simply crossed or braided, and a coil formed by winding a wire. The wire(s) may be one or more single wires or one or more stranded wires. The resin tube can be manufactured, for example, by extrusion molding. When the sheath 5 is a resin tube, the sheath 5 can be composed of a single layer or multiple layers. A part of the sheath 5 in the longitudinal direction or circumferential direction thereof may be composed of a single layer, and the other part thereof may be composed of multiple layers.

The sheath 5 can be made of, for example, a synthetic resin such as a polyolefin resin (e.g., polyethylene or polypropylene), a polyamide resin (e.g., nylon), a polyester resin (e.g., PET), an aromatic polyether ketone resin (e.g., PEEK), a polyether polyamide resin, a polyurethane resin, a polyimide resin, or a fluorine resin (e.g., PTFE, PFA, or ETFE), or a metal such as stainless steel, carbon steel, or a nickel-titanium alloy. Only one of these materials may be used alone, or two or more of these materials may be used in combination.

As shown in FIG. 8, FIG. 12, FIG. 16, FIG. 24, and FIG. 25, the puncture catheter 100 may include the handle 6. The handle 6 may have an inner cavity into which the tube 4 is inserted. The handle 6 is a portion to be gripped by the user, and it is preferable that the handle 6 has a shape that allows the handle 6 to be easily gripped by the user. It is preferable that the handle 6 is connected to a proximal end portion of the sheath 5.

The material of the handle 6 is not particularly limited, and synthetic resins including polyolefin resins such as polypropylene (PP) and polyethylene (PE), polyester resins such as polyethylene terephthalate (PET), polycarbonate resins, ABS resins, polyurethane resins, etc., can be used.

As shown in FIG. 8, FIG. 12, FIG. 16, FIG. 24, and FIG. 25, it is preferable that the puncture needle 3 is transported to the target organ in the body in a state where the puncture needle 3 is disposed in the inner cavity of the sheath 5. After transportation, it is preferable to move the position of the puncture needle 3 relative to the sheath 5 toward the distal side and cause the puncture needle 3 to protrude from a distal end portion of the sheath 5 to puncture the organ.

As shown in FIG. 13, the puncture catheter 100 may include the first tube 41. It is preferable that the above-described tube 4 includes the first tube 41.

As shown in FIG. 13 to FIG. 15 and FIG. 25 to FIG. 28, it is preferable that the first tube 41 has an inner cavity 401 extending in the longitudinal direction x of the puncture needle 3 and is connected to the proximal end portion of the puncture needle 3. It is preferable that a plurality of grooves 410 are formed in the first tube 41.

Since the helical member 20 is disposed around the tubular member 10 in the puncture needle 3 included in the puncture catheter 100, the puncture needle 3 can be easily screwed into the target tissue by rotating the puncture needle 3 about the tubular member 10 as an axis. Accordingly, it becomes easier for the puncture needle 3 to puncture the target tissue. In addition, the wire 21 constituting the helical member 20 bites into the tissue, so that it becomes difficult for the puncture needle 3 to come out of the tissue. Furthermore, the puncture depth of the puncture needle 3 can be easily adjusted by adjusting the rotation of the puncture needle 3. In addition, when a plurality of grooves 410 are formed in the first tube 41 connected to the puncture needle 3, flexibility can be imparted to the first tube 41. Moreover, when the first tube 41 in which the grooves 410 are formed is connected to the puncture needle 3, it can be made easier to transmit torque to the puncture needle 3. Therefore, the flexibility and operability of the puncture catheter 100 can be improved.

As shown in FIG. 14, FIG. 15, FIG. 27, and FIG. 28, the first tube 41 can be configured to have an inner surface 402 facing the inner cavity 401 and an outer surface 403 facing the exterior thereof. The first tube 41 has a distal end and a proximal end in the longitudinal direction x of the puncture needle 3. As for the first tube 41, it is preferable that both the distal end and the proximal end thereof are open.

The shape of the first tube 41 can be, for example, a hollow cylindrical shape, a hollow polygonal prism shape, or the like.

The length of the first tube 41 in the longitudinal direction x of the puncture needle 3 can be set to 100 mm or more, 130 mm or more, 160 mm or more, etc. The length of the first tube 41 in the longitudinal direction x of the puncture needle 3 can be set to 300 mm or less, 270 mm or less, 240 mm or less, etc. The length of the first tube 41 in the longitudinal direction x of the puncture needle 3 refers to the maximum length among lengths of the first tube 41 in the longitudinal direction x of the puncture needle 3. When the puncture needle 3 is used as a puncture catheter for puncturing the cardiac muscle, it is preferable that the length of the first tube 41 in the longitudinal direction x of the puncture needle 3 is 200 mm.

The length of the first tube 41 in the radial direction y of the puncture needle 3 can be set to 0.3 mm or more, 0.5 mm or more, 0.8 mm or more, etc. The length of the first tube 41 in the radial direction y of the puncture needle 3 can be set to 20 mm or less, 15 mm or less, 10 mm or less, etc. The length of the first tube 41 in the radial direction y of the puncture needle 3 refers to the maximum length among lengths of the first tube 41 in the radial direction y of the puncture needle 3. When the puncture needle 3 is used as a puncture catheter for puncturing the cardiac muscle, it is preferable that the length of the first tube 41 in the radial direction y of the puncture needle 3 is 0.9 mm.

As shown in FIG. 14, FIG. 15, FIG. 27, and FIG. 28, it is preferable that the inner cavity 11 of the tubular member 10 communicates with the inner cavity 401 of the first tube 41. Accordingly, a liquid such as a cell preparation or a drug solution can be delivered to the puncture needle 3 through the inner cavity 401 of the first tube 41.

The first tube 41 can be made of, for example, any of the materials exemplified as those that can form the puncture needle 3, but it is preferable that the first tube 41 is made of a metal.

The plurality of grooves 410 formed in the first tube 41 may be through grooves as shown in FIG. 15 and FIG. 28, or may be bottomed grooves as shown in FIG. 14 and FIG. 27. Both through grooves and bottomed grooves may be formed in the first tube 41. When through grooves and bottomed grooves are combined or either through grooves or bottomed grooves are selected for the grooves 410 formed in the first tube 41, it becomes easier to provide the first tube 41 with desired flexibility and rigidity.

As shown in FIG. 15 and FIG. 28, it is preferable that a first resin layer 404 made of a resin is provided on the outer surface 403 of the first tube 41. Accordingly, regardless of whether the grooves 410 formed in the first tube 41 are through grooves or bottomed grooves, a liquid such as a cell preparation or a drug solution can be delivered to the puncture needle 3 through the inner cavity 401 of the first tube 41. When grooves 410 are through grooves, it is preferable that the first resin layer 404 is provided on the outer surface 403 of the first tube 41.

The first resin layer 404 may be provided so as to cover the entire outer surface 403 of the first tube 41, or may be provided so as to cover only a part of the outer surface 403 of the first tube 41. As shown in FIG. 15 and FIG. 28, the first resin layer 404 may be provided over the grooves 410.

The first resin layer 404 can be made of, for example, a synthetic resin such as a polyolefin resin (e.g., polyethylene or polypropylene), a polyamide resin (e.g., nylon), a polyester resin (e.g., PET), an aromatic polyether ketone resin (e.g., PEEK), a polyether polyamide resin, a polyurethane resin, a polyimide resin, or a fluorine resin (e.g., PTFE, PFA, or ETFE).

As shown in FIG. 13 and FIG. 26, it is preferable that when observed in the direction perpendicular to the longitudinal direction x of the puncture needle 3, the extending directions of the grooves 410 and the longitudinal direction x of the puncture needle 3 intersect at an angle α. Each angle α at which the extending direction of each groove 410 and the longitudinal direction x of the puncture needle 3 intersect may be the same or different. The angle α may be 70° or more, 73° or more, or 75° or more. The angle α may be 85° or less, 83° or less, or 80° or less. When the angle α is set within the above range, it can be made easier to transmit torque to the puncture needle 3.

It is preferable that the length of one groove 410 in the extending direction of the groove 410 is shorter than the length of the outer circumference of the first tube 41. The length of one groove 410 in the extending direction of the groove 410 refers to the maximum length among lengths of one groove 410 in the extending direction of the groove 410.

As shown in FIG. 13 and FIG. 26, it is preferable that the plurality of grooves 410 include a first groove 411 and a second groove 412 located at a position different from that of the first groove 411, and that, when observed in the direction perpendicular to the longitudinal direction x of the puncture needle 3, the second groove 412 is located in the extending direction of the first groove 411. Accordingly, it can be made easier to transmit torque to the puncture needle 3.

As shown in FIG. 13 and FIG. 26, it is preferable that the maximum length between the first groove 411 and the second groove 412 in the extending direction of the first groove 411 is shorter than the maximum length of the first groove 411 in the extending direction of the first groove 411. Accordingly, the interval between the first groove 411 and the second groove 412 can be made relatively short, thereby making it easier to impart flexibility to the first tube 41.

As shown in FIG. 13 and FIG. 26, it is preferable that the plurality of grooves 410 include a third groove 413 located adjacent to the first groove 411 in the longitudinal direction x of the puncture needle 3, and that the maximum length between the first groove 411 and the third groove 413 in the longitudinal direction x of the puncture needle 3 is shorter than the length of the pitch P of the helical member 20. Accordingly, the interval between the first groove 411 and the third groove 413 can be made relatively short, thereby making it easier to impart flexibility to the first tube 41.

As shown in FIG. 13, it is preferable that the outer diameter of the tubular member 10 is smaller than the outer diameter of the first tube 41. Accordingly, a distal end portion of the first tube 41 has an effect of serving as a stopper when colliding with the tissue, thereby making it easier for only the puncture needle 3 to puncture the tissue. Therefore, the puncture needle 3 can be inhibited from deeply puncturing the tissue.

It is preferable that the distal end portion of the first tube 41 is connected to the puncture needle 3. The first tube 41 and the puncture needle 3 may be connected via a diameter-reducing member 30. The diameter-reducing member 30 is a member having an outer diameter decreasing toward the distal side. More specifically, as shown in FIG. 13 to FIG. 16 and FIG. 25 to FIG. 28, it is preferable that a proximal end portion of the tubular member 10 and the distal end portion of the first tube 41 are connected by the diameter-reducing member 30.

It is preferable that the diameter-reducing member 30 has an inner cavity 31 penetrating in the longitudinal direction x of the puncture needle 3. The diameter-reducing member 30 can be configured to have an inner surface facing the inner cavity 31 and an outer surface facing the exterior thereof. The diameter-reducing member 30 has a distal end and a proximal end in the longitudinal direction x of the puncture needle 3. As for the diameter-reducing member 30, it is preferable that both the distal end and the proximal end thereof are open. It is preferable that the inner cavity 31 of the diameter-reducing member 30 communicates with the inner cavity 11 of the tubular member 10 and the inner cavity 401 of the first tube 41.

The diameter-reducing member 30 can be made of, for example, any of the materials exemplified as those that can form the puncture needle 3, but it is preferable that the diameter-reducing member 30 is made of a metal. The material forming the first tube 41 and the material forming the diameter-reducing member 30 may be the same or different.

The shape of the diameter-reducing member 30 can be, for example, a hollow truncated polygonal pyramid shape, a hollow truncated cone shape, or the like.

As shown in FIG. 16 and FIG. 25, the puncture catheter 100 may include the second tube 42 that has an inner cavity extending in the longitudinal direction x of the puncture needle 3 and is connected to a proximal end portion of the first tube 41. It is preferable that the above-described tube 4 includes the first tube 41 and the second tube 42.

The second tube 42 can be configured to have an inner surface facing the inner cavity thereof and an outer surface facing the exterior thereof. The second tube 42 has a distal end and a proximal end in the longitudinal direction x of the puncture needle 3. As for the second tube 42, it is preferable that both the distal end and the proximal end thereof are open.

The shape of the second tube 42 can be, for example, a hollow cylindrical shape, a hollow polygonal prism shape, or the like.

The length of the second tube 42 in the longitudinal direction x of the puncture needle 3 can be set to 1000 mm or more, 1200 mm or more, 1400 mm or more, etc. The length of the second tube 42 in the longitudinal direction x of the puncture needle 3 can be set to 2000 mm or less, 1800 mm or less, 1600 mm or less, etc. The length of the second tube 42 in the longitudinal direction x of the puncture needle 3 refers to the maximum length among lengths of the second tube 42 in the longitudinal direction x of the puncture needle 3. When the puncture needle 3 is used as a puncture catheter for puncturing the cardiac muscle, it is preferable that the length of the second tube 42 in the longitudinal direction x of the puncture needle 3 is 1300 mm.

The length of the second tube 42 in the radial direction y of the puncture needle 3 can be set to 0.3 mm or more, 0.5 mm or more, 0.8 mm or more, etc. The length of the second tube 42 in the radial direction y of the puncture needle 3 can be set to 20 mm or less, 15 mm or less, 10 mm or less, etc. The length of the second tube 42 in the radial direction y of the puncture needle 3 refers to the maximum length among lengths of the second tube 42 in the radial direction y of the puncture needle 3. When the puncture needle 3 is used as a puncture catheter for puncturing the cardiac muscle, it is preferable that the length of the second tube 42 in the radial direction y of the puncture needle 3 is 0.9 mm.

It is preferable that a distal end portion of the second tube 42 is connected to the proximal end portion of the first tube 41. It is also preferable that the inner cavity of the second tube 42 communicates with the inner cavity 401 of the first tube 41. Accordingly, a liquid such as a cell preparation or a drug solution can be delivered to the puncture needle 3 through the inner cavity 401 of the first tube 41 and the inner cavity of the second tube 42.

The second tube 42 can be made of, for example, any of the materials exemplified as those that can form the puncture needle 3, but it is preferable that the second tube 42 is made of a metal. The material forming the first tube 41 and the material forming the second tube 42 may be the same or different.

As shown in FIG. 16 and FIG. 25, the puncture catheter 100 may include the sheath 5. It is preferable that the puncture needle 3, the first tube 41, and the second tube 42 are insertable into the inner cavity of the sheath 5. It is preferable that the puncture needle 3, the first tube 41, and the second tube 42 are movable in the longitudinal direction x of the puncture needle 3 within the inner cavity of the sheath 5.

As shown in FIG. 16 and FIG. 25, the puncture catheter 100 may include the handle 6. The second tube 42 may be inserted into the inner cavity of the handle 6.

Next, the catheter system 200 including the puncture catheter 100 including the puncture needle 3 according to one or more embodiments of the present invention will be described with reference to FIG. 25 to FIG. 28. The components already described above are denoted by the same reference characters, and the description thereof is sometimes omitted.

As shown in FIG. 25, it is preferable that the catheter system 200 includes the puncture catheter 100 including the puncture needle 3, the first tube 41, and the sheath 5.

As shown in FIG. 25, it is preferable that the sheath 5 has an inner cavity 51 into which the puncture needle 3 and the first tube 41 are inserted. The sheath 5 can be configured to have an inner surface 52 facing the inner cavity 51 and an outer surface 53 facing the exterior thereof. The sheath 5 has a distal end and a proximal end in the longitudinal direction x of the puncture needle 3. As for the sheath 5, it is preferable that both the distal end and the proximal end thereof are open. As shown in FIG. 25, the puncture needle 3, the first tube 41, and the second tube 42 may be inserted into the inner cavity 51 of the sheath 5. It is preferable that the puncture needle 3, the first tube 41, and the second tube 42 are movable in the longitudinal direction x of the puncture needle 3 within the inner cavity 51 of the sheath 5.

As shown in FIG. 25, when the length in the radial direction of the tubular member 10 between the outer surface 13 of the tubular member 10 and the portion of the wire 21 located outermost in the radial direction of the tubular member 10 is denoted by H, the maximum outer diameter of the first tube 41 is denoted by d, the maximum outer diameter of the tubular member 10 is denoted by s, and the inner diameter of the sheath 5 is denoted by D, it is preferable that the catheter system 200 satisfies a relationship of D>d>s+2H.

Since the helical member 20 is disposed around the tubular member 10 in the puncture needle 3 included in the catheter system 200, the puncture needle 3 can be easily screwed into the target tissue by rotating the puncture needle 3 about the tubular member 10 as an axis. Accordingly, it becomes easier for the puncture needle 3 to puncture the target tissue. In addition, the wire 21 constituting the helical member 20 bites into the tissue, so that it becomes difficult for the puncture needle 3 to come out of the tissue. Furthermore, the puncture depth of the puncture needle 3 can be easily adjusted by adjusting the rotation of the puncture needle 3. In addition, when the catheter system 200 satisfies the relationship of D>d>s+2H, the outer diameter of the first tube 41 is larger than the outer diameter of the puncture needle 3. Accordingly, the distal end portion of the first tube 41 collides with the tissue, it is made easier for only the puncture needle 3 to puncture the tissue, and the tissue can be inhibited from being deeply punctured.

As shown in FIG. 25, the puncture catheter 100 included in the catheter system 200 may include the second tube 42 that has an inner cavity extending in the longitudinal direction x of the puncture needle 3 and is connected to the proximal end portion of the first tube 41, as described above.

Although the disclosure has been described with respect to only a limited number of embodiments, those skilled in the art, having benefit of this disclosure, will appreciate that various other embodiments may be devised without departing from the scope of the present disclosure. Accordingly, the scope of the invention should be limited only by the attached claims.

### DESCRIPTION OF THE REFERENCE CHARACTERS

10: tubular member
10d: distal end of tubular member
10p: proximal end of tubular member
11: inner cavity of tubular member
12: inner surface of tubular member
13: outer surface of tubular member
14: hole
15: tapered portion
16: straight tube portion
17: wire-arrangement region
18: wire-non-arrangement region
20: helical member
20d: distal end of helical member
21: wire
21d: distal end of wire
22: inner surface of helical member
23: outer surface of helical member
24: connection member
25: adhesive
P: pitch of helical member
3: puncture needle
30: diameter-reducing member
31: inner cavity of diameter-reducing member
4: tube
41: first tube
401: inner cavity of first tube
402: inner surface of first tube
403: outer surface of first tube
404: first resin layer
410: groove
411: first groove
412: second groove
413: third groove
42: second tube
5: sheath
51: inner cavity of sheath
52: inner surface of sheath
53: outer surface of sheath
6: handle
100: puncture catheter
200: catheter system

1. A medical puncture needle having a longitudinal direction, the puncture needle comprising:
a tubular member having an inner cavity extending in the longitudinal direction; and
a helical member comprising a wire wound helically around the tubular member,
wherein a distal end portion of the wire and the tubular member are not fixed to each other, or the distal end portion of the wire and the tubular member are not in contact with each other.

2. The puncture needle according to claim 1, wherein a cross-sectional shape of the wire is polygonal in a cross-section perpendicular to the longitudinal direction.

3. The puncture needle according to claim 1, wherein, in a cross-section perpendicular to the longitudinal direction and passing through a midpoint of the puncture needle in the longitudinal direction, relationships of 2h<H and H−h<W are satisfied,
wherein a length in a radial direction of the tubular member between an outer surface of the tubular member and a portion of the wire located outermost in a radial direction of the tubular member is denoted by H,
a separation distance between the outer surface of the tubular member and an inner surface of the helical member is denoted by h, and
a width of the wire in a circumferential direction of the puncture needle is denoted by W.

4. The puncture needle according to claim 1, wherein the tubular member includes:
a tapered portion having an outer diameter decreasing toward a distal side; and
a straight tube portion located on a proximal side with respect to the tapered portion.

5. The puncture needle according to claim 1, configured to puncture an organ inside a body.

6. The puncture needle according to claim 1, wherein a part of the tubular member and a part of the helical member are directly fixed to each other.

7. The puncture needle according to claim 1, wherein an inner diameter of the helical member is equal to or less than twice an outer diameter of the tubular member.

8. The puncture needle according to claim 1, wherein the distal end portion of the wire has a tapered shape, and wherein the tapered shape extends from a distal end of the wire to a position away from the distal end of the wire on a proximal side by a length equal to ½ of a pitch of the helical member.

9. The puncture needle according to claim 1, wherein, in a cross-section perpendicular to the longitudinal direction and passing through a midpoint of the puncture needle in the longitudinal direction, a length of the wire in a radial direction of the tubular member is shorter than a length of the wire in a circumferential direction of the tubular member.

10. The puncture needle according to claim 1, wherein the wire has a section in which a value obtained by dividing a length of the wire in a circumferential direction of the tubular member, measured in a cross-section perpendicular to the longitudinal direction, by a length of the wire in a radial direction of the tubular member, measured in the cross-section, decreases toward a distal side.

11. A puncture catheter comprising the puncture needle according to claim 1.

12. The puncture catheter according to claim 11, further comprising a first tube having an inner cavity extending in the longitudinal direction and connected to a proximal end portion of the puncture needle, wherein
the first tube has a plurality of grooves formed therein.

13. The puncture catheter according to claim 12, wherein the first tube is made of a metal.

14. The puncture catheter according to claim 13, wherein a first resin layer made of a resin is provided on an outer surface of the first tube.

15. The puncture catheter according to claim 12, wherein an extending direction of the plurality of grooves and the longitudinal direction intersect each other at an angle α observed in a direction perpendicular to the longitudinal direction.

16. The puncture catheter according to claim 15, wherein the angle α is 70° or more and 85° or less.

17. The puncture catheter according to claim 12, wherein
the plurality of grooves include:
a first groove; and
a second groove located at a position different from a position of the first groove, and
the second groove is located on an extending direction of the first groove when observed in a direction perpendicular to the longitudinal direction.

18. The puncture catheter according to claim 17, wherein a maximum length between the first groove and the second groove in the extending direction of the first groove is shorter than a maximum length of the first groove in the extending direction of the first groove.

19. The puncture catheter according to claim 17, wherein
the plurality of grooves include a third groove located adjacent to the first groove in the longitudinal direction, and
a maximum length between the first groove and the third groove in the longitudinal direction is shorter than a length of a pitch of the helical member.

20. A catheter system comprising the puncture catheter according to claim 12,
wherein the puncture catheter further comprises a sheath having an inner cavity into which the puncture needle and the first tube are inserted, and
the catheter system satisfies a relationship of D>d>s+2H,
wherein a length in a radial direction of the tubular member between an outer surface of the tubular member and a portion of the wire located outermost in the radial direction of the tubular member is denoted by H,
a maximum outer diameter of the first tube is denoted by d,
a maximum outer diameter of the tubular member is denoted by s, and
an inner diameter of the sheath is denoted by D.
